(19) United States
(12) Patent Application Publication
(10) Pub. No.: US 2026/0294653 A1
(43) Pub. Date: Oct. 1, 2026

(54) LOCAL MANUFACTURING OF A MEDICAL STENT BASED ON A COMPUTATIONAL MODELED OUTCOME PREDICTION

(71) Applicant: KONINKLIJKE PHILIPS N.V., EINDHOVEN (NL)

(72) Inventors: René Leonardus Jacobus Marie UBACHS, EINDHOVEN (NL); Olaf VAN DER SLUIS, LANDGRAAF (NL)

(21) Appl. No.: 18/874,120

(22) PCT Filed: Jun. 20, 2023
(86) PCT No.: PCT/EP2023/066515

§ 371 Date: Dec. 12, 2024

Related U.S. Application Data
(60) Provisional application No. 63/357,035, filed on Jun. 30, 2022.

(51) Int. Cl. A61F 2/82

(52) U.S. Cl. CPC A61F 2/82; A61F 2240/002

(57) ABSTRACT

A method for additive manufacturing of a medical stent includes generating, by a processor (152) executing instructions, a model of anatomy of a subject; obtaining a computer (140)-aided design (CAD) model of a stent; simulating deployment of the stent in the anatomy of the subject; determining whether the model of the stent satisfies an optimization metric based on the simulating, and optimizing the model of the stent by repeating obtaining and the simulating until the model of the stent satisfies the optimization metric. The stent is manufactured by additive manufacturing in response to the model of the stent being optimized.

### BACKGROUND

When anatomical vessels are obstructed either by stenosis or by externally applied load, patency can be restored and maintained by deployment of a stent. Stenosis refers to the narrowing of blood vessels, whereas patency refers to the condition of being open, expanded or unobstructed. A stent is a tube for deployment into the lumen of an anatomical vessel, and is used to restore and maintain patency. The amount of patency restored is determined by the size of the stent, the amount of radial outward force used to deploy the stent and the amount of chronic outward force which the stent can supply after deployment.

Typically, a stent chosen for a targeted anatomical vessel will be oversized with respect to the targeted anatomical vessel. The choice of an oversized stent may balance forces applied by the stenotic (narrowed) vessel wall with the forces resulting from radially compressing the stent so as to maintain the desired patency. The stent essentially functions as a preloaded spring.

Anatomical vessels are typically not perfectly straight tubes with constant diameters. Instead, anatomical vessels tend to be tortuous with varying cross-sectional areas. Anatomical vessels also tend to vary for different people with different health and demographic characteristics. Accordingly, stents and other in-body devices are commonly not subject specific, and instead tend to have standardized features such as dimensions. This means that the stents and other in-body devices are incapable of providing optimal outcomes for subjects.

### SUMMARY

According to an aspect of the present disclosure, a method for additive manufacturing of a medical stent includes generating, by a processor executing instructions, a model of anatomy of a subject; obtaining a computer-aided design (CAD) model of a stent; simulating deployment of the stent in the anatomy of the subject; determining whether the model of the stent satisfies an optimization metric based on the simulating, and optimizing the model of the stent by adjusting a characteristic of the model of the stent and repeating the obtaining and the simulating until the model of the stent satisfies the optimization metric. The stent is manufactured by additive manufacturing in response to the model of the stent being optimized.

According to another aspect of the present disclosure, a tangible, non-transitory computer readable storage medium stores a computer program. The computer program, in response to being executed by a processor, causes a system to: obtain a computer-aided design (CAD) model of a stent; simulate deployment of the stent in the anatomy of the subject; determine whether the model of the stent satisfies an optimization metric based on simulating deployment of the stent, and optimize the model of the stent by adjusting a characteristic of the model of the stent and repeating obtaining the model of the stent and simulating blood flow through the stent until the model of the stent satisfies the optimization metric. The stent is manufactured by additive manufacturing in response to the model of the stent being optimized.

According to another aspect of the present disclosure, a system for additive manufacturing of a medical stent includes a memory that stores instructions; and a processor that executes the instructions. In response to being executed by the processor, the instructions cause the system to: generate a model of anatomy of a subject; obtain a computer-aided design (CAD) model of a stent; simulate deployment of the stent in the anatomy of the subject; determine whether the model of the stent satisfies an optimization metric based on simulating deployment of the stent, and optimize the model of the stent by adjusting a characteristic of the model of the stent and repeating obtaining the model of the stent and simulating blood flow through the stent until the model of the stent satisfies the optimization metric. The stent is manufactured by additive manufacturing in response to the model of the stent being optimized.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 illustrates a system for additive manufacturing of a medical stent based on a computational modeled outcome prediction, in accordance with a representative embodiment.

FIG. 2 illustrates a method for additive manufacturing of a medical stent based on a computational modeled outcome prediction, in accordance with a representative embodiment.

FIG. 3 illustrates another method for additive manufacturing of a medical stent based on a computational modeled outcome prediction, in accordance with a representative embodiment.

FIG. 4 illustrates a prediction of the shape of a deployed stent in a curved vessel for additive manufacturing of a medical stent based on a computational modeled outcome prediction, in accordance with a representative embodiment.

FIG. 5 illustrates a prediction of stresses in a vessel wall due to shape contact for additive manufacturing of a medical stent based on a computational modeled outcome prediction, in accordance with a representative embodiment.

FIG. 6 illustrates a computer system, on which a method for additive manufacturing of a medical stent based on a computational modeled outcome prediction is implemented, in accordance with another representative embodiment.

### DETAILED DESCRIPTION

In the following detailed description, for the purposes of explanation and not limitation, representative embodiments disclosing specific details are set forth in order to provide a thorough understanding of embodiments according to the present teachings. However, other embodiments consistent with the present disclosure that depart from specific details disclosed herein remain within the scope of the appended claims. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. Definitions and explanations for terms herein are in addition to the technical and scientific meanings of the terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

As used herein, the term “substantially” means to within acceptable limits or degree to one of ordinary skill in the art.

Unless otherwise noted, when an element or component is said to be “connected to”, “coupled to another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below.

As described herein, stents may be customized based on the anatomy of subjects so as to provide optimal outcomes for subjects. The stents may be manufactured local to the subjects using additive manufacturing. Computational modelling may be employed to predict the shape of a deployed stent in an anatomical vessel based on the geometry of the anatomical vessel. The computational modelling may allow assessment of various clinical outcome parameters, such as patency, blood flow, and wall shear stress. The inputs for the calculations in the computational modelling may include geometric properties and material properties of the stent and geometric properties and material properties of the subject in which the stent is to be deployed. Using inverse modeling techniques in combination with topology optimization, the initial shape of the stent may be optimized such that after implantation the optimal outcome for an individual subject is made more likely.

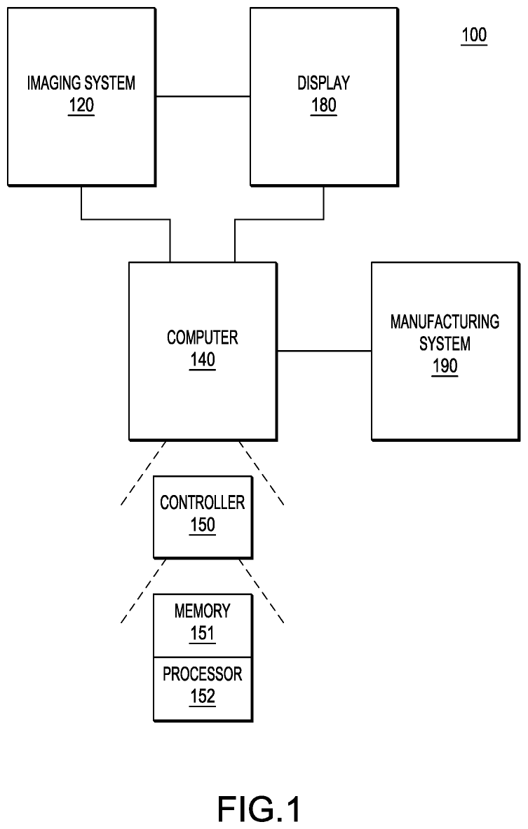

FIG. 1 illustrates a system for additive manufacturing of a medical stent based on a computational modeled outcome prediction, in accordance with a representative embodiment.

The system 100 is a system for additive manufacturing of a medical stent based on a computational modeled outcome prediction, and includes components that may be provided together or that may be distributed. The system 100 includes an imaging system 120, a computer 140, a display 180, and a manufacturing system 190.

The imaging system 120 is used for radiology, and may be, for example, an X-ray system, a magnetic resonance imaging (MRI) system, an ultrasound system, an endoscopy system, a computerized tomography (CT) system, or any other form of an imaging system that is consistent with the teachings herein.

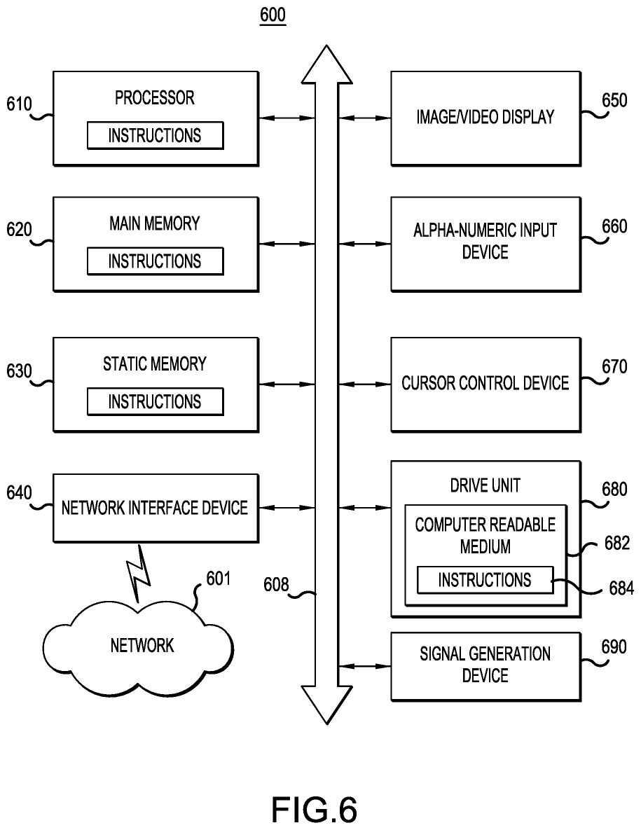

The computer 140 includes at least a controller 150, and the controller 150 includes at least a memory 151 that stores instructions and a processor 152 that executes the instructions. A computer that can be used to implement the computer 140 is depicted in FIG. 6, though a computer 140 may include more or fewer elements than depicted in FIG. 1 or FIG. 6. In some embodiments, multiple different elements of the system 100 in FIG. 1 may include a controller such as the controller 150.

The controller 150 may include interfaces, such as a first interface, a second interface, a third interface, and a fourth interface. One or more of the interfaces may include ports, disk drives, wireless antennas, or other types of receiver circuitry that connect the controller 150 to other electronic elements. One or more of the interfaces may also include user interfaces such as buttons, keys, a mouse, a microphone, a speaker, a display separate from the display 180, or other elements that users can use to interact with the controller 150 such as to enter instructions and receive output.

The controller 150 may perform some of the operations described herein directly and may implement other operations described herein indirectly. For example, the controller 150 may indirectly control other operations such as by generating and transmitting content to be displayed on the display 180. The controller 150 may directly control other operations such as logical operations performed by the processor 152 executing instructions from the memory 151 based on input received from electronic elements and/or users via the interfaces. Accordingly, the processes implemented by the controller 150 when the processor 152 executes instructions from the memory 151 may include steps not directly performed by the controller 150.

The display 180 is connected to the imaging system 120 and the computer 140 in FIG. 1. The display 180 may be local to the imaging system 120 and/or the computer 140, or may be remotely connected to the imaging system 120 and/or the computer 140. The display 180 may be connected to the imaging system 120 and/or the computer 140 via local wired interfaces such as Ethernet cables or via local wireless interfaces such as Wi-Fi connections. The display 180 may be interfaced with other user input devices by which users can input instructions, including mouses, keyboards, thumbwheels and so on. The display 180 may be a monitor such as a computer monitor, a display on a mobile device, an augmented reality display, a television, an electronic whiteboard, or another screen configured to display electronic imagery. The display 180 may also include one or more input interface(s) that may connect to other elements or components, as well as an interactive touch screen configured to display prompts to users and collect touch input from users. In some embodiments, multiple different displays such as the display 180 may be provided together or separately for the imaging system 120 and the computer 140.

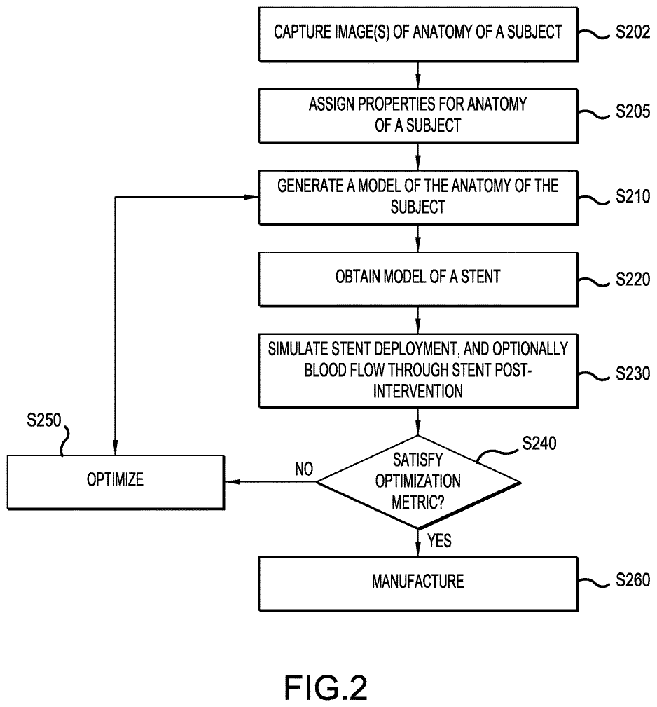

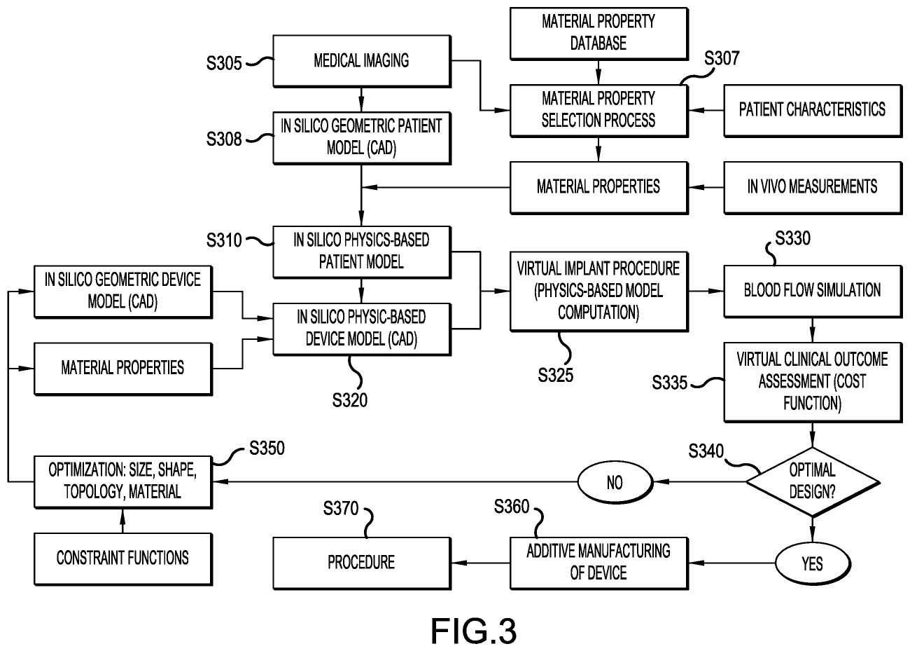

The manufacturing system 190 is representative of a local manufacturing system that can dynamically manufacture a stent using, for example, an additive manufacturing process. Additive manufacturing is or includes 3D printing, which involves construction of a three-dimensional object from a model such as a digital 2D or 3D CAD model. Other known manufacturing processes suitable for local operation may also be used. Just by way of illustration, the present teaching contemplate milling laser cutting or milling as alternative manufacturing methods. The manufacturing system 190 may use an optimized digital 3D model of a stent resulting from the methods of FIG. 2 and FIG. 3 as an input to generate the stent. Additive manufacturing may be performed by a process in which material is deposited, joined or solidified under computer control, with material being added together. Additive manufacturing by the manufacturing system 190 may involves adding materials layer by layer by deposition, joining or solidification under the control of a computer. A computer for the manufacturing system 190 may be the computer 140, or may be an entirely separate computer (not shown). A computer for the manufacturing system 190 may execute software to process the optimized digital 3D model and control, for example, one or more lasers in the manufacturing system 190 to melt a metal power to produce an optimized metal stent. A computer for the manufacturing system 190 may execute software to process the optimized digital 3D model and control, for example, a stereolithography (SLA) 3D printer with a laser to cure thermosetting liquid resins into hardened process in a photopolymerization process.

FIG. 2 illustrates a method for additive manufacturing of a medical stent based on a computational modeled outcome prediction, in accordance with a representative embodiment.

The method of FIG. 2 may be performed by the system 100 including imaging system 120, the computer 140 with the controller 150, the display 180 and the manufacturing system 190.

The method of FIG. 2 starts by capturing one or more image(s) of anatomy of a subject at S202. The image(s) of the anatomy of the subject may be captured by a medical imaging system such as the imaging system 120 in FIG. 1. The medical image(s) may be X-ray images, echocardiogram images, electrocardiogram images (ECK/EKG), or other types of medical images. Additionally, the image(s) of the anatomy of the subject may be captured at S202 before an intervention where the stent will be deployed in a subject, or towards the beginning of an intervention where the stent will be deployed in a subject. In other words, the imaging may be performed at the same facility and in the same timeframe as the deployment of the stent, or at a different facility and before the time or even the day when the stent is to be deployed.

At S205, properties for the anatomy of the subject are assigned. As an example, the properties for the anatomy of the subject may be assigned by the computer 140 in FIG. 1 executing an image analysis program and processing demographic and health history data of the subject. The properties for the anatomy may be assigned fully or in part based on health condition and/or demographic information of the subject, such as gender, age, preexisting condition(s) and so on. In some embodiments, the properties for the anatomy may be assigned based on clinically-obtained data of the subject, such as based on the image(s) of the anatomy of the subject captured at S202, and/or based on blood tests, saliva tests, DNA tests, weight, blood pressure readings, pulse oximetry readings, and other types of clinical readings of a subject.

At S210, a model of the anatomy of the subject is generated. The model of the anatomy of the subject may be generated by the computer 140 in FIG. 1. For example, each image captured at S202 may be segmented to obtain a mask of one or more pixels for each object present in the image. A 3D model may be generated based on the segmentations from the medical images.

At S220, a model of a stent is obtained. The model of the stent may be obtained by the computer 140 in FIG. 1. For example, the first time S220 is performed, the model may be generated based solely on the properties for the anatomy assigned at S205, but subsequent performances of S220 may involve adjustments of the initial model and the immediately-previous model in an optimization process based on simulations described herein.

At S230, placement of the stent in the anatomy of the subject is simulated. In some embodiments, S230 also includes simulating blood flow through the stent. The simulation at S230 may be performed by the computer 140 in FIG. 1. The simulating at S230 may comprise calculating an outcome of placing the stent in the anatomy of the subject. The calculating may comprise obtaining expected motion and deformation of the stent once placed in the anatomy of the subject and subject to blood flow through the stent deployed in the vessel of the anatomy.

The calculating may also comprise subjecting the new state of the stented anatomy to expected physiological loadings, such as, but not limited to, caused by the movement of body parts and beating of the heart.

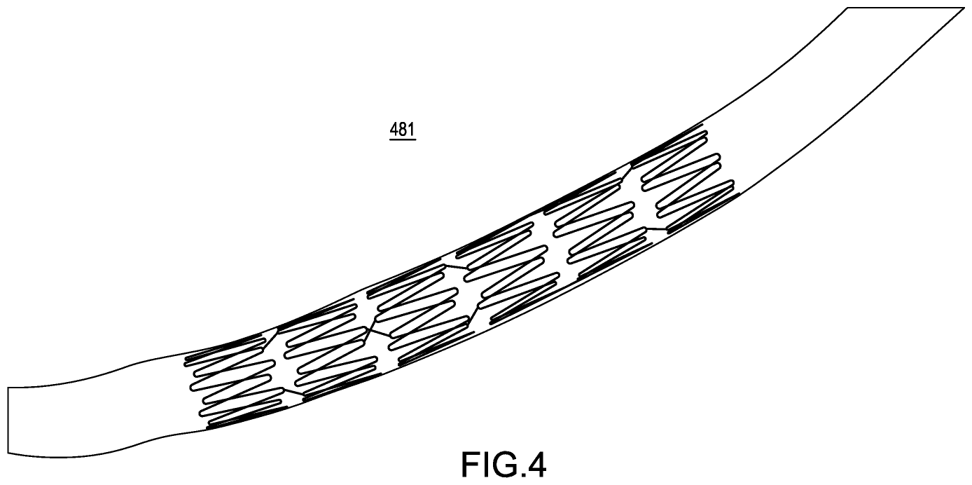

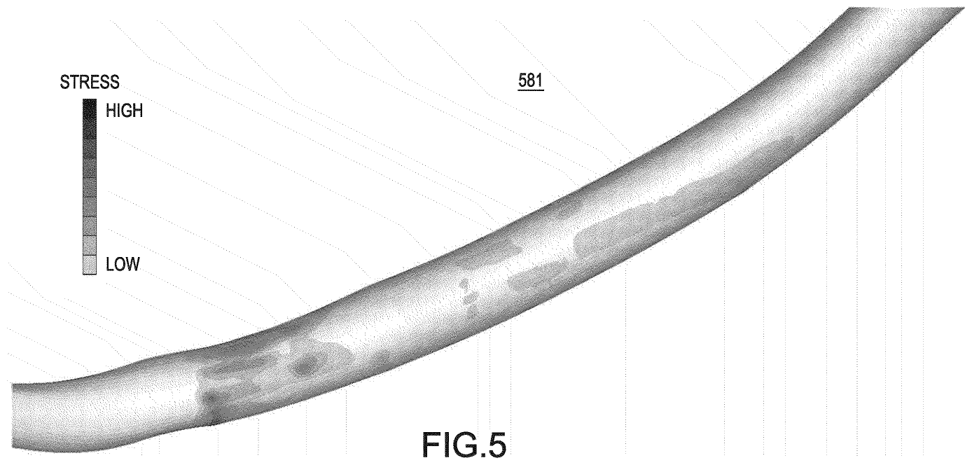

Examples of the simulated placement of the stent in the anatomy of the subject are shown in FIG. 4 and FIG. 5. FIG. 4 shows a prediction of the shape of a deployed stent in a coronary vessel obtained through computational modeling. FIG. 5 shows predicted stresses in a vessel wall due to stent contact. In addition to predicting of stress in the vessel, other metrics are contemplated by the present teachings. These other contemplated predicted measures include, but are not limited to strain, and contact force. Moreover, these predicted metrics may be applied not only to the stent as noted, but also to the anatomy of the subject. Specifically, deformation and reaction forces of the stent, the vessel, and/or other anatomical tissue follow from the same computations because the forces are substantially at equilibrium. As such, the methods of the present teachings are contemplated for application to the deformed shape of any vessel, and/or other anatomical tissue to determine the stresses, strains and forces. In performing these predictions, the equations of the model are adjusted to account for the behavior of the materials.

At S240, the method of FIG. 2 includes determining whether the model of the stent satisfies an optimization metric. The determination at S240 may be based on the simulation at S230, and may be performed by the computer 140 in FIG. 1. The optimization metric used for S240 may be based on any one or more of a variety of characteristics of the anatomy and/or the stent. For example, the optimization metric may be based on mechanical stress on the anatomy of the subject. The optimization metric may be based on expected patency of a vessel in which the stent is to be placed after the stent is placed. The optimization metric may be based on expected durability of the stent. The optimization metric may be based on expected blood flow through a vessel of the anatomy in which the stent is to be placed after the stent is placed. The optimization metric may be based on one or more of these examples, or other relevant characteristics of the anatomy and/or the stent. For example, the optimization metric may comprise a weighted combination of multiple different optimization metrics such as multiple of the examples noted above. If the optimization metric is not satisfied (S240=No), at S250 one or more optimization adjustments are determined and the process returns to S210. The optimization adjustments may be to one or more characteristic features of the model of the stent, and may be made based on the simulation at S230.

Example adjustments may include increasing or decreasing diameter of the stent at one location, multiple locations or along the entire length of the stent. Example adjustments may also or alternative include changing a material of the stent, such as from plastic to metal or from plastic to metal, or from one type of metal to another type of metal or from one type of plastic to another type of plastic. Other adjustments for optimization are contemplated. For example, adjustment of the parameters may include changing of the stent strut dimensions, overall or locally or changing the length of the stent, adding or removing parts of a stent. In case of braided stents adjustments to the parameters related to the pitch angle, wire thickness or shape, wire amount, for example, are also contemplated. Adjustments can also be made to adapt to branches from or bifurcations of the vessel, or introducing and/or adjusting an axial curvature to the stent.

The method of FIG. 2 may include repeating the obtaining and the simulating until the model of the stent satisfied the optimization metric after an adjustment to the model of the stent. The model of the anatomy of the subject may be repeatedly obtained for each time the obtaining and simulating are repeated. The repeating may include minimizing a cost function by calculating a weighted average of the parameters of an outcome of the simulating for each simulation and comparing the weighted average of parameters of the outcome of the simulating for each simulation to an optimization metric. The repeating may also or alternatively include minimizing a cost function until a minimization results in an improvement falling below a threshold such as a predetermined threshold.

If the optimization metric is satisfied (S240=Yes) at S260 the stent is manufactured by an additive manufacturing process or by an alternative manufacturing process which can be performed locally.

FIG. 3 illustrates another method for additive manufacturing of a medical stent based on a computational modeled outcome prediction, in accordance with a representative embodiment.

The method of FIG. 3 includes labelled elements for steps and functions along with some labelled elements for inputs. The labelled elements for steps and functions are designed with element numbers corresponding to the steps and functions.

The method of FIG. 2 may be performed by the system 100 including imaging system 120, the computer 140 with the controller 150, the display 180 and the manufacturing system 190.

At S305, medical imaging is performed. The medical imaging may be performed by the imaging system 120 in FIG. 1. Medical imaging at S305 may include X-ray imaging, echocardiogram imaging, electrocardiogram imaging (EKG/ECG), and/or any other type of medical imaging appropriate for preparing deployment of stents in subjects. The medical images may be subject to post-processing, such as segmenting to delineate objects in the medical images and image recognition algorithms for labelling anatomical objects in the medical images. In some embodiments, the medical imaging at S305 may be performed at the beginning of a procedure that includes the deployment of the optimized stent which results from the method of FIG. 3.

At S307, a material property selection process is performed. The material property selection process may be based on material properties from a material property database, subject characteristics such as demographic information, and medical images from the medical imaging at S305. The material properties reflect characteristics of the anatomy of the subject and are used to generate an in silico physics-based patient model at S310. In other words, the reference to material property for inputs to S310 in FIG. 3 refers to materials characteristics of the anatomy. Examples of these material properties may include elasticity of artery walls, viscosity of blood, relevant diameters or cross-sectional areas of an artery around the location where a stent will be deployed, and more. Material properties of anatomy may be expected to vary based on demographic characteristics such as age, gender, height and weight. The material properties of anatomy may also vary based on health history such as preexisting conditions, as well as measurable health characteristics such as blood pressure.

The selected material properties from the material property selection process at S307 are combined with in vivo measurements. The combination of the selected material properties and in vivo measurements (if any) may include a set of characteristics of the relevant anatomy of the subject. In turn, the characteristics correspond to known physical relationships that describe how the anatomy may respond to deployment of a stent, such as how a vessel wall will respond to being pushed and how the flow of blood will respond to the expansion of diameter in a vessel.

At S308, an in silico geometric patient model is generated via computer-aided design (CAD). The in silico geometric patient model is generated at S308 based on the medical images from the medical imaging at S305. For example, segmentation may be performed on each medical image to represent anatomical features in each medical image, and the geometric patient model may be generated based on the segmentation. Segmentation involves dividing an image into pixels or groups/regions of pixels to generate masks for objects in the image.

At S310, an in silico physics-based patient model is generated. The in silico physics-based patient model is generated at S310 based on the in silico geometric patient model generated at S308 along with the selected material properties and the in vivo measurements. The properties and measurements may be fit to one or more equations or algorithms to generate the model at S310. For example, equations may describe the behavior of blood flow, tissue response, and other anatomical characteristics given the properties and measurements obtained as inputs to S310.

At S320, initially a first version of an in silico physics-based device model is generated via computer-aided design (CAD). The in silico physics-based device model is generated at S320 based on the in silico geometric patient model from S308 and the material properties. The result of S320 is a computed final configuration of the stent to be deployed in the vessel. During the following optimization process additional information is directed into S320 which is used to adjust the in silico physics-based device model into an improved second version. This process may be repeated iteratively.

At S325, a virtual implant procedure is performed as a physics-based model computation. The virtual implant procedure is performed at S325 based on the in silico physics-based patient model from S310 and the in silico physics-based device model from S320. The virtual implant procedure may include a simulated deployment of the device model from S320 to the patient model from S310, so as to determine exact 3D coordinates of the device model and the relevant vessel in which the stent is to be deployed. The computation at S325 may include calculating metrics of parameters relevant to clinical outcome such as stress and strain in the vessel wall, stress and strain in the stent, and damage to the vessel wall. In addition, the stented anatomy may be subjected virtually to physiological loading conditions such as originating from body (part) or organ movements.

At S330, a blood flow simulation may optionally be performed. The blood flow simulation at 330 involves computing vessel stresses and blood flow characteristics in the treated vessel with the presence of the deformed stent in the vessel. The computation at S330 may include calculating metrics of parameters relevant to a clinical outcome such as wall shear stress, turbulence, mean and local flow velocity and pressure drop, and these metrics may be used as inputs to a cost function at S335. The wall shear stress may be calculated for multiple locations in the area of the vessel where the stent will be deployed.

Examples of the simulated stresses in the vessel wall based on placement of the stent in the anatomy of the subject are shown in FIG. 4 and FIG. 5. FIG. 4 shows a prediction of the shape of a deployed stent in a curved vessel obtained through computational modeling. FIG. 5 shows predicted stresses in a vessel wall due to stent contact.

At S335, a virtual clinical outcome assessment is performed as a cost function based on the blood flow simulation at S330. The metrics calculated at S330 may be used as terms in the cost function and hence driving design optimization for the stent. A cost function is determined consisting of a scaled and weighted average of the relevant clinical outcome parameters.

Optimization of the device design is then directed by minimizing this cost function. This cost function may be defined prior to construction of the computational model. The weighing factors of the cost function are introduced to find the desired tradeoffs between conflicting objectives.

For example, a cost function might be constructed to maximize patency while minimizing stresses in the vessel wall. Obviously, maximizing patency will lead to a high stiffness stent design with a large amount of material. This will however lead to high stresses in the vessel wall and obstruction and/or disturbance of the blood flow.

The cost function performed at S335 may impose a mix of absolute limits and relative calculations. For example, absolute limits may be imposed for anatomical characteristics that are deemed unacceptable on an absolute basis. Relative calculations may consider several anatomical characteristics and stent characteristics together, such as in a polynomial. As an example, a particular material for a stent such as metal may be optimal for vessels in a male over 50 with a relative high cholesterol reading. Another relative calculation may compare a measured lumen diameter to an expected lumen diameter for a typical or average subject with the demographic characteristics of the subject.

At S340, a determination is made as to whether the in silico physics-based device model generated at S320 is an optimal design. The determination at S340 is made based on the virtual clinical outcome assessment at S335. The determination at S340 may include obtaining one or more metric(s) based on the simulating at S330, and determining whether the metric(s) meet absolute thresholds or relative thresholds. For example, the determination at S340 when performed a second or later time may include determining whether any improvement has resulted from an optimization adjustment to the stent model, or whether any resultant improvement is above a predetermined threshold such as a 1% improvement.

If the in silico physics-based device model is an optimal design (S340=Yes), local manufacturing of the stent is performed at S360, and the stent is implanted in a procedure at S370. The manufacturing of the stent may be performed by the manufacturing system 190 in FIG. 1, and is performed only once an optimal model of the stent is confirmed at S340.

If the in silico physics-based device model is not an optimal design (S340 =No), optimization is performed at S350. The optimization at S350 is a structural optimization step or process, and is used to generate an updated in silico physics-based device model at S320. For example, a gradient decent method may be used at S350 to minimize the cost function assessed at S335. The specific design changes in terms of geometry may be achieved by optimization of size, shape, topology or one or more other types of physical characteristics.

The optimization at S350 is based on constraint functions, and may involve optimization of the size, shape, topology, material and/or other characteristics of the stent. The constraint functions are introduced to maintain limits on the design of the stent. The constraint functions may include realistic physical limits for materials such as potential stiffness, dimensional limits for the stent such as maximum length, minimum length or other dimensions, and other types of physical limits.

An in silico geometric device model is re-generated based on the optimization. The re-generated in silico geometric device model and the material properties of the stent are again used to generate an in silico physics-based device model at S320 and the process repeated until an optimal design is found for the stent. Using this new design, the process after S310 may be repeated until the cost function assessed at S335 is sufficiently minimized. The cost function assessed at S335 is sufficiently maximized when, for example, a specified limit is reached, or when no further minimization is possible on an absolute basis and/or on a relative basis compared to a predetermined threshold.

The result of performing the method of FIG. 3 once or as many times as required to optimize the design of the in silico physics-based device model is the production of the stent. The produced stent is the result of computational optimization, and is produced through additive manufacturing at S360. The produced stent may then be loaded into a delivery system, and implanted in the subject at S370.

In a first set of additional embodiments supplemental or alternative to FIG. 3, the design of the stent to be optimized may be described by a parametrized computational model of the stent. The design parameters may be variables that can be adapted within predetermined bounds such as known physical bounds. The design parameters may include strut thickness, strut length, local stent diameter and/or other design parameters. In the first set of additional embodiments, the gradients of the cost function with respect to the available parameters may be determined and optimization may be performed by following the gradients down to the minimum cost function value.

In a second set of additional embodiments supplemental or alternative to FIG. 3, a parametrized stent design may be used. A design of experiments (DOE) of the design of the stent may be performed, and the results may be used to find the optimum design. In the second set of additional embodiments, a screening or fractional factorial design of experiments may be used before the construction of a response surface model or the calculation of a full factorial design of experiments, so that an optimal design is found.

In a third set of additional embodiments supplemental or alternative to FIG. 3, an initial stent design may be created which is optimized using shape and topology optimization. In the third set of additional embodiments, a computer program may be used to strategically remove and add material from and to the device design so as to minimize the cost function.

FIG. 4 illustrates a prediction of the shape of a deployed stent in a curved vessel for additive manufacturing of a medical stent based on a computational modeled outcome prediction, in accordance with a representative embodiment.

As an example, the predicted shape of a deployed stent in FIG. 4 may be obtained at S230 in FIG. 2 or at S330 in FIG. 3 as part of the simulation. That is, simulations may include deployment of the stent, the blood flow through the deployed stent, and other characteristics such as reactions of the vessel to the deployment of the stent.

FIG. 5 illustrates a prediction of stresses in a vessel wall due to contact with a medical stent manufactured based on a computational modeled outcome prediction, in accordance with a representative embodiment.

In FIG. 5, variations in stresses in a vessel wall due to deployment of a stent may be distinguished as a heat map or another form of color map where different colors and tones are used to illustrate different stresses in the vessel wall. As an example, the predicted stresses in the vessel wall in FIG. 5 may also be obtained at S230 in FIG. 2 or at S330 in FIG. 3 as part of the simulation. Simulations are not limited to the blood flow through the stent, and instead may encompass a variety measurements and calculations for multiple different aspects of the result of deploying a stent in a vessel of a subject's anatomy.

FIG. 6 illustrates a computer system, on which a method for additive manufacturing of a medical stent based on a computational modeled outcome prediction is implemented, in accordance with another representative embodiment.

FIG. 6 illustrates a computer system, on which a method for additive manufacturing of a medical stent based on a computational modeled outcome prediction is implemented, in accordance with another representative embodiment.

Referring to FIG. 6, the computer system 600 includes a set of software instructions that can be executed to cause the computer system 600 to perform any of the methods or computer-based functions disclosed herein. The computer system 600 may operate as a standalone device or may be connected, for example, using a network 601, to other computer systems or peripheral devices. In embodiments, a computer system 600 performs logical processing based on digital signals received via an analog-to-digital converter.

In a networked deployment, the computer system 600 operates in the capacity of a server or as a client user computer in a server-client user network environment, or as a peer computer system in a peer-to-peer (or distributed) network environment. The computer system 600 can also be implemented as or incorporated into various devices, such as a workstation that includes a controller, a stationary computer, a mobile computer, a personal computer (PC), a laptop computer, a tablet computer, or any other machine capable of executing a set of software instructions (sequential or otherwise) that specify actions to be taken by that machine. The computer system 600 can be incorporated as or in a device that in turn is in an integrated system that includes additional devices. In an embodiment, the computer system 600 can be implemented using electronic devices that provide voice, video or data communication. Further, while the computer system 600 is illustrated in the singular, the term “system” shall also be taken to include any collection of systems or sub-systems that individually or jointly execute a set, or multiple sets, of software instructions to perform one or more computer functions.

As illustrated in FIG. 6, the computer system 600 includes a processor 610. The processor 610 may be considered a representative example of a processor of a controller and executes instructions to implement some or all aspects of methods and processes described herein. The processor 610 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 610 is an article of manufacture and/or a machine component. The processor 610 is configured to execute software instructions to perform functions as described in the various embodiments herein. The processor 610 may be a general-purpose processor or may be part of an application specific integrated circuit (ASIC). The processor 610 may also be a microprocessor, a microcomputer, a processor chip, a controller, a microcontroller, a digital signal processor (DSP), a state machine, or a programmable logic device. The processor 610 may also be a logical circuit, including a programmable gate array (PGA), such as a field programmable gate array (FPGA), or another type of circuit that includes discrete gate and/or transistor logic. The processor 610 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 600 further includes a main memory 620 and a static memory 630, where memories in the computer system 600 communicate with each other and the processor 610 via a bus 608. Either or both of the main memory 620 and the static memory 630 may be considered representative examples of a memory of a controller, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 620 and the static memory 630 are articles of manufacture and/or machine components. The main memory 620 and the static memory 630 are computer-readable mediums from which data and executable software instructions can be read by a computer (e.g., the processor 610). Each of the main memory 620 and the static memory 630 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, Blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 600 further includes a video display unit 650, such as a liquid crystal display (LCD), an organic light emitting diode (OLED), a flat panel display, a solid-state display, or a cathode ray tube (CRT), for example. Additionally, the computer system 600 includes an input device 660, such as a keyboard/virtual keyboard or touch-sensitive input screen or speech input with speech recognition, and a cursor control device 670, such as a mouse or touch-sensitive input screen or pad. The computer system 600 also optionally includes a disk drive unit 680, a signal generation device 690, such as a speaker or remote control, and/or a network interface device 640.

In an embodiment, as depicted in FIG. 6, the disk drive unit 680 includes a computer-readable medium 682 in which one or more sets of software instructions 684 (software) are embedded. The sets of software instructions 684 are read from the computer-readable medium 682 to be executed by the processor 610. Further, the software instructions 684, when executed by the processor 610, perform one or more steps of the methods and processes as described herein. In an embodiment, the software instructions 684 reside all or in part within the main memory 620, the static memory 630 and/or the processor 610 during execution by the computer system 600. Further, the computer-readable medium 682 may include software instructions 684 or receive and execute software instructions 684 responsive to a propagated signal, so that a device connected to a network 601 communicates voice, video or data over the network 601. The software instructions 684 may be transmitted or received over the network 601 via the network interface device 640.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, additive manufacturing of a medical stent based on a computational modeled outcome prediction enables customized manufacturing of devices such as stents using additive manufacturing. The teachings herein may be used in instances where a stent or a stent-like device is used in the venous or arterial system, the neurovascular system or esophageal and gastric system. The resultant devices may be used to maintain patency, repair dissection, positioning of a valve, etc. The teachings herein may also be employed in supporting stents around artificial valves such as transcatheter aortic or mitral valves.

Although additive manufacturing of a medical stent based on a computational modeled outcome prediction has been described with reference to several exemplary embodiments, it is understood that the words that have been used are words of description and illustration, rather than words of limitation. Changes may be made within the purview of the appended claims, as presently stated and as amended, without departing from the scope and spirit of additive manufacturing of a medical stent based on a computational modeled outcome prediction in its aspects. Although additive manufacturing of a medical stent based on a computational modeled outcome prediction has been described with reference to particular means, materials and embodiments, additive manufacturing of a medical stent based on a computational modeled outcome prediction is not intended to be limited to the particulars disclosed; rather additive manufacturing of a medical stent based on a computational modeled outcome prediction extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept.

Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A method for additive manufacturing of a medical stent, comprising:
generating, by a processor executing instructions, a model of anatomy of a subject;
obtaining a computer aided design (CAD) model of a stent;
simulating deployment of the stent in the anatomy of the subject;
determining whether the model of the stent satisfies an optimization metric based on the simulating, and
optimizing the model of the stent by adjusting a characteristic of the model of the stent and repeating the obtaining and the simulating until the model of the stent satisfies the optimization metric, wherein the stent is manufactured by additive manufacturing in response to the model of the stent being optimized.

2. The method of claim 1, further comprising:
simulating blood flow through the stent when placed in the anatomy of the subject,
wherein the stent is customized based on the model of the anatomy of the subject.

3. The method of claim 1, wherein the optimization metric is based on at least one of mechanical stress on the anatomy of the subject, expected patency of a vessel in which the stent is to be placed after the stent is placed, expected durability of the stent, and expected blood flow through a vessel in which the stent is to be placed after the stent is placed.

4. The method of claim 1, wherein the optimization metric comprises a weighted combination of multiple different optimization metrics.

5. The method of claim 1, further comprising:
generating an image of the anatomy of the subject to generate the model of the anatomy of the subject.

6. The method of claim 5, further comprising:
converting the image of the anatomy of the subject to the model of the anatomy of the subject, wherein the model of the anatomy of the subject is configured to describe a response of the anatomy of the subject under mechanical loading.

7. The method of claim 6, further comprising: assigning properties for the anatomy based on health condition and demographic information of the subject.

8. The method of claim 7, wherein the properties are assigned based on clinically-obtained data of the subject.

9. The method of claim 1, wherein the simulating comprises calculating an outcome of placing the stent in the anatomy of the subject.

10. The method of claim 9, wherein the calculating comprises obtaining expected motion and deformation of the stent once placed in the anatomy of the subject.

11. The method of claim 9, wherein repeating the generating, the obtaining and the simulating until the model of the stent satisfies the optimization metric comprises minimizing a cost function by calculating a weighted average of parameters of an outcome of the simulating for each simulation and comparing the weighted average of parameters of the outcome of the simulating for each simulation to an optimization metric.

12. The method of claim 9, wherein repeating the generating, the obtaining and the simulating until the model of the stent satisfies the optimization metric comprises minimizing a cost function until a minimization results in an improvement falling below a threshold.

13. The method of claim 1, further comprising: changing at least one of size, shape, topology, a material, or property of a material of the model of the stent before repeating the simulating.

14. A tangible non-transitory computer readable storage medium that stores a computer program, wherein the computer program, in response to being executed by a processor, causes a system to:
generate a model of anatomy of a subject;
obtain a computer aided design (CAD) model of a stent;
simulate deployment of the stent in the anatomy of the subject;
determine whether the model of the stent satisfies an optimization metric based on simulating deployment of the stent, and
optimize the model of the stent by adjusting a characteristic of the model of the stent and repeating obtaining the model of the stent and simulating blood flow through the stent until the model of the stent satisfies the optimization metric, wherein the stent is manufactured by additive manufacturing in response to the model of the stent being optimized.

15. The tangible non-transitory computer readable storage medium of claim 14, wherein the computer program, in response to being executed by a processor, further causes the system to:
simulate blood flow through the stent when placed in the anatomy of the subject,
wherein the optimization metric is based on at least one of mechanical stress on the anatomy of the subject, expected patency of a vessel in which the stent is to be placed after the stent is placed, expected durability of the stent, and expected blood flow through a vessel in which the stent is to be placed after the stent is placed.

16. A system for additive manufacturing of a medical stent, comprising:
a memory that stores instructions; and
a processor that executes the instructions, wherein, in response to being executed by the processor the instructions cause the system to:
generate a model of anatomy of a subject;
obtain a computer aided design (CAD) model of a stent;
simulate deployment of the stent in the anatomy of the subject; and
determine whether the model of the stent satisfies an optimization metric based on simulating deployment of the stent, and
optimize the model of the stent by adjusting a characteristic of the model of the stent and repeating obtaining the model of the stent and simulating blood flow through the stent until the model of the stent satisfies the optimization metric, wherein the stent is manufactured by additive manufacturing in response to the model of the stent being optimized.

17. The system of claim 16, wherein, in response to being executed by the processor, the instructions further cause the system to:
simulate blood flow through the stent when placed in the anatomy of the subject,
wherein the optimization metric is based on at least one of mechanical stress on the anatomy of the subject, expected patency of a vessel in which the stent is to be placed after the stent is placed, expected durability of the stent, and expected blood flow through a vessel in which the stent is to be placed after the stent is placed.
